(19) United States
(12) Patent Application Publication
(10) Pub. No.: US 2026/0174165 A1
(43) Pub. Date: Jun. 25, 2026

(54) COOLING NECK BAND

(71) Applicant: Chang Geun AN, Busan (KR)

(72) Inventor: Chang Geun AN, Busan (KR)

(21) Appl. No.: 18/845,859

(22) PCT Filed: Dec. 27, 2023
(86) PCT No.: PCT/KR2023/021727

§ 371 Date: Sep. 10, 2024

(30) Foreign Application Priority Data
Aug. 8, 2023 (KR) 10-2023-0103720

(51) Int. Cl. A41D 23/00; A41D 1/00; A41D 20/00; A45F 5/02; A61F 5/01; A61F 5/02; A61F 5/055; A61F 5/058; A61F 7/00; A61F 7/02; A61F 7/08; A61F 13/15; A61F 13/49; A61F 13/496; A61F 13/514; A61F 13/515; A61F 13/536; A61F 13/56

(52) U.S. Cl. CPC A41D 23/00; A41D 1/002; A41D 20/00; A45F 5/021; A61F 5/0111; A61F 5/026; A61F 5/055; A61F 5/05816; A61F 7/00; A61F 7/086; A61F 13/15593; A61F 13/49003; A61F 13/49014; A61F 13/49015; A61F 13/49017; A61F 13/4963; A61F 13/51464; A61F 13/515; A61F 13/536; A61F 13/5622; A61F 2007/0007; A61F 2007/0098; A61F 2007/0276

(57) ABSTRACT

Provided is a cooling neck band. The cooling neck band is easily worn around the neck of a wearer, is not easily dislodged by prolonged use or vigorous movement, and maintains a cool sensation on the neck for an extended time.

### TECHNICAL FIELD

The present disclosure relates to a cooling neck band and, more particularly, to a cooling neck band that may be easily worn around the neck of a wearer, may not be easily dislodged by prolonged use or vigorous movement, and may maintain a cool sensation on the neck of the wearer for an extended period of time.

### BACKGROUND ART

In general, heat stroke may occur on hot summer days when a large amount of heat accumulates in the body due to high outdoor temperatures, or when a worker works for long hours in an industrial site involving thermal energy.

To prevent such accidents caused by heat, ice packs filled with ice or frozen wet towels or frozen water bottles from a freezer may be placed on body parts, such as the face and neck, to provide a cool sensation on the body parts.

There are problems, however, in that ice in ice packs, which are used to temporarily cool the body, may easily melt, thereby requiring frequent ice replacement, and cold towels, which may need to be repeatedly placed in and removed from a freezer to keep cold, are also problematic.

In addition to the problem of condensation caused by the ice in the ice pack melting and the water from the cold towel wetting clothes, there was also a problem that the temperature difference between the ice in the ice pack or the cold towel frozen in the freezer and the temperature of the human body is great, thereby causing an abnormal symptom such as headache when the ice pack or the cold towel is in contact with the body or is placed around the neck.

To solve this problem, Patent Document 1 proposes an ice neck band using a phase change material (PCM). The ice neck band has a configuration including: a band attachment member that may be worn in a wrapped form; an ice pack embedded with a phase change material and disposed on one side of the band attachment member to absorb heat generated by a body of a person and make the person feel cool; a pack protection member combined with an attachment member for attaching and detaching the ice pack to and from the band attachment member; and cold air leakage prevention members coupled to the upper and lower portions of the one side of the band attachment member to prevent the cold air of the ice pack from leaking to the outside. The neck band may be easily worn on the neck of the person and may provide a cooling sensation for an extended period of time by maintaining the cold transmitted to the neck of the person wearing the neck band at a predetermined temperature for an extended period of time.

However, it is necessary for the band attachment member to be attached or tied to the neck with Velcro to detachably wear the ice neck band on the neck, and the rough side of the Velcro may have the side effect of rubbing the skin, and the ice neck band may easily come loose or fall off the body during prolonged wearing.

### DISCLOSURE

### Technical Problem

The present disclosure is intended to overcome the problems of the related art, and an objective of the present disclosure is to provide a cooling neck band including a ring-shaped band having elasticity and a phase change material (PCM) embedded in the band, the cooling neck band being able to be easily worn around the neck of a wearer, not to be easily dislodged during prolonged use or vigorous movement, and to maintain a cooling sensation on the neck of the wearer for an extended period of time.

### Technical Solution

To realize at least one of the above-described objectives, provided is a cooling neck band including: an elastic band for elastically and closely fitting around the neck of a wearer; and a cooling band detachably provided on an inner side of the elastic band, and including cooling cubes each having a PCM embedded in a first side thereof to deliver a cold sensation to the neck of the wearer by direct contact, wherein the elastic band is formed of a plastic material plastically processed into a horseshoe-shaped plate having a predetermined length, with a portion of a forward-facing side thereof being removed in a shape that wraps around the neck of the wearer, such that the elastic band is wearable on and detachable from the neck by an effort of the wearer by using elastic portions extending from opposite sides of a neck portion of the elastic band configured to wrap around the neck of the wearer, and the cooling band has an integral body formed by fusion, the integral body including: a first film formed of thermoplastic polyurethane and having a length corresponding to the length of the elastic band; and a second film formed of thermoplastic polyurethane and having a length corresponding to the length of the first film, wherein the plurality of cooling cubes protrude from the second film, are equally spaced apart from each other in a longitudinal direction of the second film, and each comprise a phase change material embedded therein.

The plastic material of the elastic band may include one or a combination of polyethylene, polypropylene, vinyl chloride resin, vinyl acetate resin, polystyrene, acrylonitrile butadiene styrene resin, and acrylic resin.

The cooling band may include a plurality of belt loops provided on a side facing away from the cooling cubes and spaced apart from each other in a longitudinal direction of the cooling band, with the elastic band being coupled the belt loops in a belt-type manner.

The elastic band may include bent portions on opposite ends to prevent the cooling band coupled in a belt-type manner by the belt loops from slipping away in the longitudinal direction.

The cooling band may include closure pockets on opposite ends thereof to receive portions of opposite ends of the elastic band to prevent the cooling band from slipping away from the elastic band.

### Advantageous Effects

According to the present disclosure having the above-described configuration, the cooling neck band may be easily worn around the neck of a wearer, may not be easily dislodged during prolonged use or vigorous movement, and may maintain a cooling sensation on the neck of the wearer for an extended period of time.

### DESCRIPTION OF DRAWINGS

FIG. 1 is a perspective view illustrating a cooling neck band according to a first embodiment of the present disclosure;

FIG. 2 is an exploded perspective view illustrating the cooling neck band according to the first embodiment of the present disclosure;

FIG. 3 is a perspective view illustrating a cooling neck band according to a second embodiment of the present disclosure;

FIG. 4 is an exploded perspective view illustrating the cooling neck band according to the second embodiment of the present disclosure; and

FIGS. 5 and 6 are illustrate cross-sectional and mounted states of a cooling band used in the cooling neck band according to the present disclosure.

### BEST MODE

In the following description of the present disclosure, detailed descriptions of known functions and components incorporated herein will be omitted to avoid rendering the subject matter of the present disclosure unclear. Terms used herein are defined in light of the functions of the present disclosure, which may change depending on user, operator's intention, or common practice. Definitions should therefore be taken in the context of the entire specification. The terms used in the detailed description are intended only to describe embodiments of the present disclosure and should not be construed as being limiting. Unless expressly used otherwise, expressions in the singular form include the meaning of the plural form. In this description, expressions such as “including” or “comprising” are intended to refer to certain features, numbers, steps, actions, elements, parts or combinations thereof, and should not be construed to exclude the existence or possibility of one or more other features, numbers, steps, actions, elements, parts or combinations thereof other than those described.

In each of the systems illustrated in the drawings, elements in some instances may have the same or different reference numerals to suggest that the elements shown may be different or similar. However, elements may have different implementations and may work with any or all of the systems shown or described herein. The various elements illustrated in the drawings may be the same or different. Which one is referred to as the first element or the second element is arbitrary.

As used herein, references to a component “transmitting,” “delivering,” or “providing” data or signals to another component include transmitting data or signals from one component directly to another component, as well as transmitting data or signals to another component through at least one other component.

Hereinafter, an exemplary embodiment will be described in detail with reference to the accompanying drawing so that a person having ordinary knowledge of the present disclosure belonging to the same technical field can easily practice the exemplary embodiment.

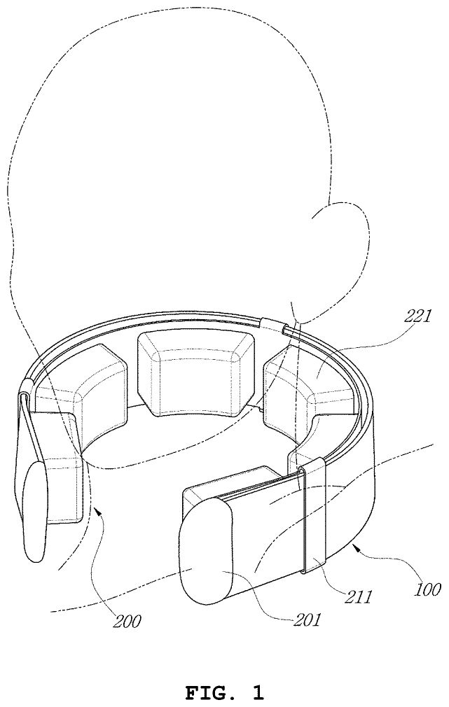

As shown in FIGS. 1 through 6, a cooling neck band according to the present disclosure is configured as an integral body such that a cooling band 200 may be removably attached to an elastic band 100 so as to be worn around the neck of a wearer to provide cooling to the wearer.

A phase change material (PCM) is a material that store heat or release stored heat through a physical change process in which a material changes from one state to another, such as solid to liquid, liquid to solid, liquid to gas, gas to liquid, etc.

Hereinafter, the phase change material will be referred to as PCM, and the configuration and geometry of the cooling neck band will be described in respective embodiments applied.

### First Embodiment

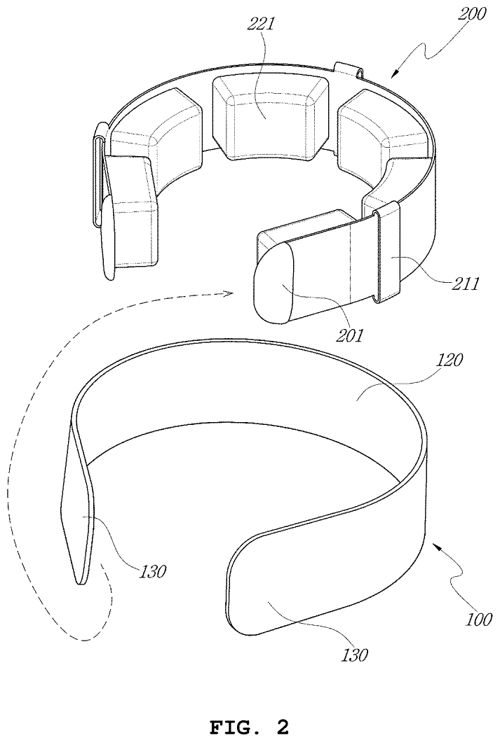

Referring to FIGS. 1 and 2, the elastic band 100 may be formed of an elastic plastic so as to closely fit around the neck of a wearer.

In other words, the elastic plastic of the elastic band 100 may representatively be a plastic belonging to a thermoplastic resin, which may be applied as one or a combination of polyethylene, polypropylene, vinyl chloride resin, vinyl acetate resin, polystyrene, acrylonitrile butadiene styrene (ABS) resin, acrylic resin, and the like. It is desirable for such a material to provide a composition which is harmless to humans and which is economical.

In particular, the elastic band 100 may be plastically processed into a plate shape having a predetermined length, and more particularly, into a “horseshoe” shape, with a portion of the forward-facing side being removed in a shape that wraps around the neck of the wearer, and may be worn on and detached from the neck by the effort of the wearer by using elastic portions 130 extending from opposite sides of a neck portion 120 configured to wrap around the neck of the wearer.

In this case, the elastic band 100 may desirably be standardized in sizes of various lengths depending on the neck of the wearer.

In addition, the cooling band 200 is detachably provided on the inner side of the elastic band 100, and has cooling cubes 221 each having a PCM 223 embedded in one side thereof to deliver a cold sensation to the neck of the wearer by direct contact.

Here, the cooling band 200 may be formed by integrally bonding, by fusion, a first film 210 and a second film 220, the width of each of which is equal to or less than the width of the elastic band 100.

Here, the first film 210 is formed of thermoplastic polyurethane (TPU), and has a length corresponding to that of the elastic band 100.

In addition, the second film 220 formed of a TPU material has a length corresponding to that of the first film 210, and a plurality of cooling cubes 221 each embedded with the PCM 223 may protrude therefrom while being equally spaced apart from each other in the longitudinal direction of the second film 220.

Here, a plurality of belt loops 211 may be provided on a side of the cooling band 200 facing away from the cooling cubes 221 and spaced apart from each other in the longitudinal direction of the cooling band 200, and the elastic band 100 may be coupled to the belt loops 211 in a belt-type manner.

In other words, by continuously providing the belt loops 211 of the cooling band 200 in the longitudinal direction of the elastic band 100 so that the cooling band 200 is positioned inside the neck portion 120 of the elastic band 100, the cooling cubes 221 of the cooling band 200 may be in direct contact with the neck of the wearer to provide a cold sensation to the neck of the wearer using the properties of the PCM 223.

In addition, while being united into an integral body by the belt loops 211, the cooling band 200 is provided with closure pockets 201 on opposite ends thereof to receive portions of the opposite ends of the elastic band 100 to prevent the cooling band 200 from slipping away from the elastic band 100, so that the cooling band 200 may not slip away from the elastic band 100 even when the wearer moves vigorously.

### Second Embodiment

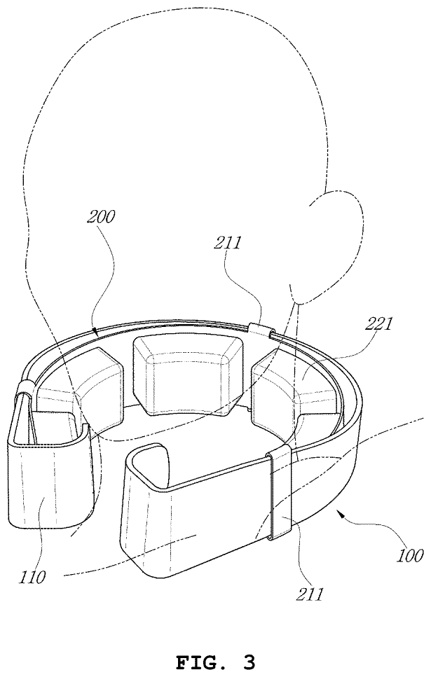

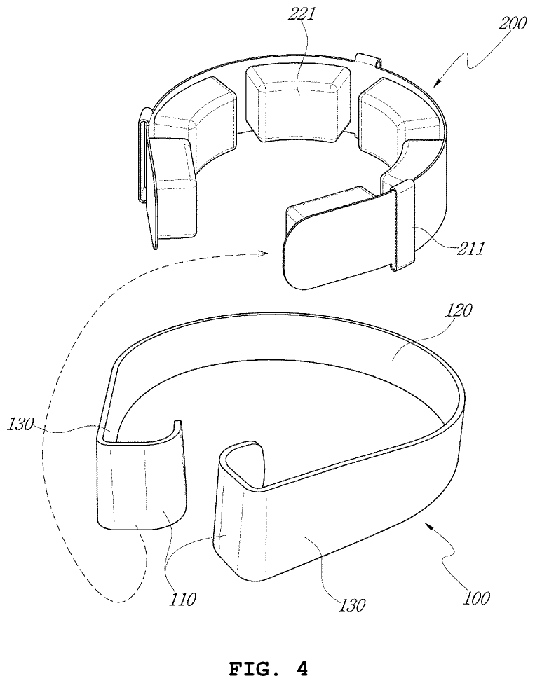

Referring to FIGS. 3 and 4, the elastic band 100 may be formed of an elastic plastic so as to closely fit around the neck of a wearer.

In other words, the elastic plastic of the elastic band 100 may representatively be a plastic belonging to a thermoplastic resin, which may be applied as one or a combination of polyethylene, polypropylene, vinyl chloride resin, vinyl acetate resin, polystyrene, ABS resin, acrylic resin, and the like. It is desirable for such a material to provide a composition which is harmless to humans and which is economical.

In particular, the elastic band 100 may be plastically processed into a plate shape having a predetermined length, and more particularly, into a “horseshoe” shape, with a portion of the forward-facing side being removed in a shape that wraps around the neck of the wearer, and may be worn on and detached from the neck by the effort of the wearer by using elastic portions 130 extending from opposite sides of a neck portion 120 configured to wrap around the neck of the wearer.

In this case, the elastic band 100 may desirably be standardized in sizes of various lengths depending on the neck of the wearer.

In addition, the cooling band 200 is detachably provided on the inner side of the elastic band 100, and has cooling cubes 221 each having a PCM 223 embedded in one side thereof to deliver a cold sensation to the neck of the wearer by direct contact.

Here, the cooling band 200 may be formed by integrally bonding, by fusion, a first film 210 and a second film 220, the width of each of which is equal to or less than the width of the elastic band 100.

Here, the first film 210 is formed of a TPU material, and has a length corresponding to that of the elastic band 100.

In addition, the second film 220 formed of a TPU material has a length corresponding to that of the first film 210, and a plurality of cooling cubes 221 each embedded with the PCM 223 may protrude therefrom while being equally spaced apart from each other in the longitudinal direction of the second film 220.

Here, a plurality of belt loops 211 may be provided on a side of the cooling band 200 facing away from the cooling cubes 221 and spaced apart from each other in the longitudinal direction of the cooling band 200, and the elastic band 100 may be coupled to the belt loops 211 in a belt-type manner.

In other words, by continuously providing the belt loops 211 of the cooling band 200 in the longitudinal direction of the elastic band 100 so that the cooling band 200 is positioned inside the neck portion 120 of the elastic band 100, the cooling cubes 221 of the cooling band 200 may be in direct contact with the neck of the wearer to provide a cold sensation to the neck of the wearer using the properties of the PCM 223.

In addition, the cooling band 200 is united into an integral body by the belt loops 211, and bent portions 110 are provided on opposite ends of the elastic band 100 or the ends of the elastic portions 130 to prevent the cooling band 200 coupled in a belt-type manner by the belt loops 211 from slipping away from the elastic band 100 in the longitudinal direction, so that the cooling band 200 may not slip away from the elastic band 100 even when the wearer moves vigorously.

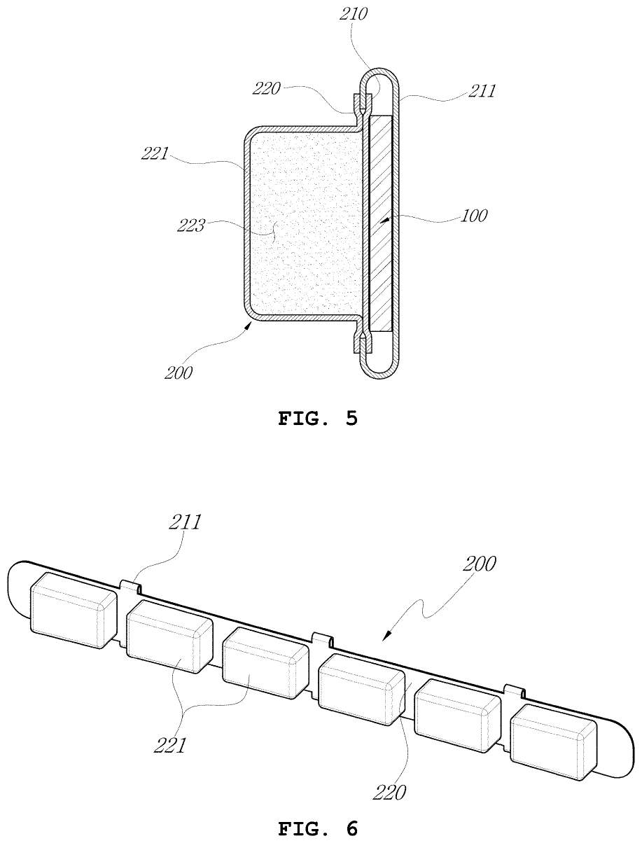

Furthermore, as shown in FIGS. 5 and 6, the first film 210 and the second film 220 of the cooling band 200 may fusion bonded to form an integral body, in which the cooling cubes 221 are formed on the second film 220 in the longitudinal direction to protrude from the inner surface of the second film 220 so that spaces for receiving the PCMs 223 therein are formed by plastically processing the second film 220. The first film 210 may be fusion bonded to the opposite side of the second film 220 to close open portions. In addition, in the bonding process, it is desirable for the belt loops 211 to be fusion bonded to edge portions of the first film 210 at intervals in the longitudinal direction, such that the elastic band 100 may be coupled thereto in a belt-type manner.

The terms and words used in this specification and claims described above are not to be construed in their ordinary or dictionary sense, but should be interpreted in a sense and concept consistent with the technical idea of the invention, based on the principle that the inventor may properly define the concept of a term to best describe his/her invention.

Therefore, it is to be understood that the configurations illustrated in the drawing and embodiments described herein are merely one best example embodiment of the invention and are not exhaustive of the technical ideas of the invention so that there may be various equivalents and variations which may be substituted for them at the time of filing.

### INDUSTRIAL APPLICABILITY

The present disclosure provides usefulness such as being able to be easily worn around the neck of a wearer, not being easily dislodged during prolonged use or vigorous movement, and being able to maintain a cooling sensation on the neck of the wearer for an extended period of time.

1. A cooling neck band comprising:
an elastic band for elastically and closely fitting around the neck of a wearer; and
a cooling band detachably provided on an inner side of the elastic band, and comprising cooling cubes each having a phase change material embedded in a first side thereof to deliver a cold sensation to the neck of the wearer by direct contact,
wherein the elastic band is formed of a plastic material plastically processed into a horseshoe-shaped plate having a predetermined length, with a portion of a forward-facing side thereof being removed in a shape that wraps around the neck of the wearer, such that the elastic band is wearable on and detachable from the neck by an effort of the wearer by using elastic portions extending from opposite sides of a neck portion of the elastic band configured to wrap around the neck of the wearer, and
the cooling band comprises an integral body formed by fusion, the integral body comprising:
a first film formed of thermoplastic polyurethane and having a length corresponding to the length of the elastic band; and
a second film formed of thermoplastic polyurethane and having a length corresponding to the length of the first film, wherein the plurality of cooling cubes protrude from the second film, are equally spaced apart from each other in a longitudinal direction of the second film, and each comprise a phase change material embedded therein.

2. The cooling neck band of claim 1, wherein the cooling band comprises a plurality of belt loops provided on a side facing away from the cooling cubes and spaced apart from each other in a longitudinal direction of the cooling band, with the elastic band being coupled the belt loops in a belt-type manner.

3. The cooling neck band of claim 2, wherein the elastic band comprises bent portions on opposite ends to prevent the cooling band coupled in a belt-type manner by the belt loops from slipping away in the longitudinal direction.

4. The cooling neck band of claim 2, wherein the cooling band comprises closure pockets on opposite ends thereof to receive portions of opposite ends of the elastic band to prevent the cooling band from slipping away from the elastic band.

5. The cooling neck band of claim 1, wherein the plastic material of the elastic band comprises one or a combination of polyethylene, polypropylene, vinyl chloride resin, vinyl acetate resin, polystyrene, acrylonitrile butadiene styrene resin, and acrylic resin.
